(19) United States
(12) Patent Application Publication
(10) Pub. No.: US 2013/0110248 A1
(43) Pub. Date: May 2, 2013

(54) LOCKABLE IMPLANT MEMBERS

(71) Applicant: ARTHRODISC, L.L.C., Park City, UT (US)

(72) Inventor: Richard I. Zipnick, Park City, UT (US)

(73) Assignee: ARTHRODISC, L.L.C., Park City, UT (US)

(21) Appl. No.: 13/667,565

(22) Filed: Nov. 2, 2012

Related U.S. Application Data
(60) Provisional application No. 61/554,589, filed on Nov. 2, 2011.
(60) Provisional application No. 61/554,600, filed on Nov. 2, 2011.
(60) Provisional application No. 61/554,616, filed on Nov. 2, 2011.

(51) Int. Cl. A61F 2/44

(52) U.S. Cl. CPC A61F 2/4455
USPC 623/17.16; 29/445

(57) ABSTRACT

An implant can include: a first implant body having one or more locking tabs and a second implant body having one or more locking receptacles containing the locking tabs of the first implant body so as to couple the first implant body with the second implant body. The first implant body can include a plurality of locking tabs received into a plurality of locking recesses. The locking tabs can be interlocking tabs that have tab heads with cross-sectional profiles larger than tab stems, and the locking receptacles are interlocking receptacles with shapes that conform with the interlocking tabs. The first implant body and second implant body can be coupled in a sandwich format, in a train format, or in a clamshell format. The first implant body can include a top portion and bottom portion that receive the second implant body therebetween.

### CROSS-REFERENCE TO RELATED APPLICATIONS

This patent application claims the benefit of U.S. Provisional Application Ser. No. 61/554,589 filed Nov. 2, 2011, U.S. Provisional Application Ser. No. 61/554,600 filed Nov. 2, 2011, and U.S. Provisional Application Ser. No. 61/554,616 filed Nov. 2, 2011, which provisional applications are each incorporated herein by specific reference in their entirety.

### BACKGROUND

An intervertebral disc is a soft tissue compartment connecting the vertebral bones in a spinal column. Each healthy disc consists of two parts, an outer annulus fibrosus (hereinafter “the annulus”) and an inner nucleus pulposus (hereinafter “the nucleus”). The annulus completely circumscribes and encloses the nucleus. The annulus is connected to an adjacent associated pair of vertebrae by collagen fibers.

The intervertebral disc is an example of a soft tissue compartment adjoining first and second bones (vertebrae) having an initial height and an initial width. Other joints consisting of a soft tissue compartment adjoining at least first and second bones having an initial height and an initial width include the joints of the hand, wrist, elbow, shoulder, foot, ankle, knee, hip, etc.

Typically, when a disc is damaged, the annulus ruptures and the nucleus herniates. Discectomy surgery removes the extruded nucleus, leaving behind the ruptured annulus. The ruptured annulus is, by itself, ineffective in controlling motion and supporting the loads applied by the adjacent pair of vertebrae. With time, the disc flattens, widens, and bulges, compressing nerves and producing pain. Uncontrolled loads are transmitted to each vertebra. Each vertebra tends to grow wider in an attempt to distribute and compensate for higher loads. When a vertebra grows, bone spurs form. The bone spurs further compress nerves, producing pain.

A variety of expandable intervertebral devices are disclosed in the art to replace the intervertebral disc. Such devices are implanted intermediate an adjacent pair of vertebrae, and function to assist the vertebrae. These devices do not assist the intervertebral disc. In fact, in many cases the disc is removed.

Prior art intervertebral devices are either static or dynamic. A static intervertebral device eliminates motion. Static devices are generally square, rectangular, trapezoidal, or box shapes that are immobile. Static devices replace the disc to facilitate bone fusion. The insertion of a static device requires near-total removal of the disc. An adjacent pair of vertebrae ordinarily is contoured to the static device and a bone graft. A static device temporarily maintains the vertebrae immobilized until the bone graft heals. Static devices may, on insertion, initially expand, but their final state is immobile. Core elements with the threads on one portion reversed or oppositely wound from threads on another portion have been frequently utilized to expand immobilization (fusion) devices.

Dynamic devices are configured to be capable of moving. Inserting a dynamic device, such as a total disc prosthesis, requires a near-total removal of disc tissue. A dynamic device ordinarily is inserted to contour to the vertebral bones without a bone graft. Usually the vertebral bones are contoured to the dynamic device. Round, curved, or circular-shaped devices inserted after removing disc tissue or vertebral bone tend to migrate in the intervertebral disc space or subside within the vertebral bone. Dynamic devices are often permanent devices that replace a disc, connect vertebral bones together, and allow movement. Dynamic devices initially may expand. Also, their final state can be mobile.

Other dynamic devices require partial removal of disc tissue. These devices can be inserted within the interior (nucleus) of an intervertebral disc and contour to the vertebral bones. Nucleus devices are generally smaller than devices used as a total disc prosthesis. Nucleus devices often are single-part locking mechanisms. Fixation generally is not used and the device typically migrates within the disc space or subsides in vertebral bones. Other dynamic devices do not have a solid bearing surface, but include liquid or gas.

Other devices and methods function to patch or seal a disc without substantially supporting the vertebra. Inserting these devices requires the removal of disc tissue. These devices are usually added to the annulus, which causes widening of the annulus, and the device increases the risk of contacting the nerves of the spinal column when the disc is compressed. Still other devices can form a physical barrier with the annulus in order to function, where such a barrier positioned within the annulus can prevent the annulus from healing. Still other devices change the material property of the disc.

Therefore, there remains a need in the art for improved devices and methods for treating injuries, deformations, or other defects in any of the intervertebral discs of the spine.

### SUMMARY

In one embodiment, an implant can include: a first implant body having one or more locking tabs; and a second implant body having one or more locking receptacles containing the locking tabs of the first implant body so as to couple the first implant body with the second implant body. The first implant body can include a plurality of locking tabs received into a plurality of locking recesses. The locking tabs can be interlocking tabs that have tab heads with cross-sectional profiles larger than tab stems, and the locking receptacles are interlocking receptacles with shapes that conform with the interlocking tabs. The first implant body and second implant body can be coupled in a sandwich format or in a train format or in a clamshell format. The first implant body can include a top portion and bottom portion that receive the second implant body therebetween. The second implant body can include one or more locking tabs received into one or more locking receptacles of the first implant body. The implant can include a top surface and bottom surface both with omega-shaped recesses. The implant can include a third implant body coupled to one of the first or second implant body by corresponding interlocking tabs and interlocking receptacles having the interlocking tabs. The implant can include at least one shuttle recess extending from a top surface and/or bottom surface, the shuttle recess including a shuttle member having a porous bone or metal material. The first implant body can include a material different from the second implant body. The implant can include an endplate having fastener apertures attached to an end of the implant. The implant can include a central conduit extending from a front end to a back end. The second implant body can have one or more interlocking tabs that are interlocked with the one or more locking tabs of the first implant body. The implant can include at least one of the locking tabs or locking receptacles that have a chamfered end. The locking tab can have a slit and forms two adjacent tab portions that can be pressed together. The locking tab can extend from a side of a body of the first implant member and is pivotal with respect to the body. The locking tab can include at least one retractable protrusion, and the locking receptacle has a receptacle for the retractable protrusion, and/or the locking tab can include at least one fixed protrusion, and the locking receptacle has a receptacle for the fixed protrusion. The first implant member and second implant member can be pivotally linked through a hinge. The locking tab and locking receptacle can include corresponding ratchet members.

In one embodiment, a method is provided for assembling a multi-member implant. The method can include: providing the first implant member and second implant member; introducing the locking tab into the locking receptacle; and coupling the first implant member and second implant member by inserting a head of the locking tab into a cavity of the locking receptacle. In one aspect, the locking tab is snap-fit pressed into the locking receptacle. In another aspect, the locking tab is configured as a rail and the locking receptacle is configured as a locking slot, and the rail is slid in the slot by sliding the first implant relative to the second implant in the direction of the rail and slot.

### BRIEF DESCRIPTION OF THE FIGURES

The foregoing and following information as well as other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings, in which:

FIG. 1A includes a perspective view that illustrates an embodiment of an implant having multiple members coupled together in a sandwich format through locking tabs and tab receptacles.

FIG. 1B includes a cross-sectional view that illustrates an embodiment of the implant of FIG. 1A with optional endplate and fillers.

FIG. 1C includes a top view that illustrates the implant of FIG. 1A.

FIG. 2 includes a side view that illustrates an embodiment of an implant having multiple members coupled together in a sandwich format through locking tabs and tab receptacles.

FIG. 3A includes a perspective back view that illustrates an embodiment of an implant having multiple members coupled together in a sandwich format through locking tabs and tab receptacles.

FIG. 3B includes a perspective front view that illustrates an embodiment of an implant having multiple members coupled together in a sandwich format through locking tabs and tab receptacles.

FIG. 4 includes a side view that illustrates an embodiment of a clamshell that can open and receive and couple to a member therein to form a multi-member implant, such as the implant of FIG. 2.

FIG. 5 includes a side view that illustrates an embodiment of a method for snap-fit coupling of multiple members together in a train format through locking tabs and tab receptacles to form an implant.

FIG. 6A includes a side view that illustrates an embodiment of a method for coupling multiple members together in a train format to form an implant.

FIG. 6B includes a side view that illustrates an embodiment of a method for insert-and-rotate coupling of multiple members together in a train format to form an implant.

FIG. 7 includes a side view that illustrates an embodiment of a method for snap-fit coupling multiple members together in a train format to form an implant.

FIG. 8A includes a side view that illustrates an embodiment of a method for snap-fit coupling multiple members together in a train format to form an implant.

FIG. 8B includes a side view that illustrates an embodiment of an implant formed by the snap-fit coupling method of FIG. 8A.

FIG. 9 includes a perspective view that illustrates an embodiment of an implant having a slidable interlocking rib cam.

FIG. 10 includes a cross-sectional view that illustrates an embodiment of a method for slidable interlocking tab and tab receptacles to form an implant.

FIGS. 11A-11C include cross-sectional views that illustrate embodiments of couplable implant members that couple together in a train format to form a linked implant.

FIGS. 12A-12C include cross-sectional views that illustrate embodiments of implants having multiple members with ratchet coupling members that are coupled together in a ratcheted train format.

FIGS. 13A-13C include cross-sectional views that illustrate different tab receptacle shapes.

FIGS. 14A-14B include cross-sectional views that illustrate two alternative expandable locking mechanisms for coupling multiple members together.

FIGS. 15A-15F include side views that illustrate alternative embodiments of hingedly-linked implant members that can be unlocked to freely rotate or coupled together through locking tabs and tab receptacles to be fixed relative to each other implant member.

FIG. 16A includes a cross-sectional view that illustrates an embodiment of an implant having a cam-operable snap-fit coupling system.

FIG. 16B includes a cross-sectional view that illustrates an embodiment of offset ratchet teeth.

FIG. 17 includes a cross-sectional view of a jigsaw puzzle coupling configuration for an interlocking implant.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Generally, the present invention relates to implants, such as intervertebral implants, that include two or more members that can be coupled together by a tab being received into a receptacle. The implant can be formed by separate implant members that are not linked together, which become linked together by inserting the tab into the receptacle. Alternatively, the separate implant members may be loosely linked, such as by a hinge or wire, and then become fixedly linked by inserting the tab into the receptacle. In another alternative, separate implant members that are capable of moving in three dimensions relative to each other can be fixed in one or more dimensions by inserting the tab into the receptacle, which by way of example can result in an implant with two bodies that are rotatable or pivotal with respect to each other via the coupling of the tab and receptacle.

The multi-membered implants can be configured for therapeutic methods to manipulate and revitalize a spinal column disc. The implant devices and methods of implantation may be configured for minimizing or preventing the removal of disc material for implantation into the disc. The implantation method allows for an implant device to be inserted in the disc either through a pre-existing rupture or through an opening formed in the front, back, or sides of the disc. Increasing the space between the vertebrae bounding the disc or removing disc material can be performed, but often is not necessary to insert the implant device into the disc. The implant device can be designed to selectively provide contact with the surfaces of one or both adjacent vertebrae, and thereby selectively apply internal traction or other forces acting on the vertebrae to alter the shape of the disc. However, the implant device may only contact one vertebral surface and disc material on the other side between the vertebra and the implant surface in order to apply forces to the vertebral surfaces. The implantation of the implant device can alter the shape of the disc to withdraw herniations or bulges inwardly in order to relieve pressure on nerves adjacent the disc. Also, the shape of the disc can also be altered to draw nuclear hernias back into the interior of the disc and to produce a disc shape that improves functioning of the disc. The invention also relates to methods of manufacture, implantation, extraction, or other uses of the implant devices.

In one embodiment, the implant device is fabricated by coupling separate housing members of different materials or moduli of elasticity through tabs and corresponding tab receptacles. The tabs and tab receptacles can be shaped similar to jigsaw puzzle-shaped tabs and tab receptacle coupling members. However, the coupled housing members may be of the same material with the same modulus of elasticity.

In one embodiment, the implant device can include two or more independent pieces that can be locked together, and optionally unlocked from each other. As such, one implant body can have a locking tab that is received into a locking receptacle of the other implant. The locking tab and locking receptacle can be cooperatively shaped as jigsaw puzzle pieces that couple together. The independent pieces can be referred to as implant members or implant bodies, which can be coupled together in sandwich or train formats via locking tabs and receptacles that have corresponding shapes.

In one embodiment, an implant can be configured for deposition in the spine between two adjacent vertebrae with one implant member having a reversibly deformable tab that is received into a correspondingly-shaped receptacle on another implant member for locking and unlocking two implant members together. The locking of the tab and receptacle can provide for a fixed joint or a movable joint of the implant. The tab can be configured with at least one protuberance on at least a first elastic member which is captured by at least a corresponding receptacle on a second member to form a joint between the first and second members. The second member can include a receptacle configured as a recess, groove, slit or other feature capable of receiving the tab and/or protuberance. The reversibly deformable tab can be prepared with a material that is sufficient for elastic recovery of the implant from a locked position to an unlocked position, and vice versa. The material of the implant can provide for locking and unlocking a movable joint of the implant or adjacent implant members. The implant can include a plurality of implant members that are linked together with the reversible deformable tab and the implants can include a recess or other indentation so that the implant components can be linked together in a sandwich format or train format. The train format can include fixed joints or ball joints that allow for adjacent implant members to pivot with respect to each other. Alternatively, the tab can be substantially rigid and the body defining the receptacle can be deformable so as to receive the substantially rigid tab. In another alternative, both the tab and body defining the receptacle can be deformable.

In one embodiment, the implant can be expanded along at least one axis to separate the vertebrae. The expansion can be by opening a clam-shaped member and/or by sandwich stacking of multiple members. The expansion may also be by rotation of a cam as described in the incorporated references. As such, the implant can have a rotatable cam that can selectively expose an engaging surface through an opening in the implant, where the engaging surface can engage a vertebral surface or disc material between a vertebral surface and the engaging surface. Accordingly, the implants described herein can include the cam mechanisms described in the incorporated references. In one aspect, the implant can be locked in an expanded configuration. The implant can be in a locked expanded configuration that moves in at least one direction. The implant can be in a locked expanded configuration that is fixed. The implant can be configured to arcuately expand.

In one embodiment, a hinge can be created by at least one tab (e.g., extrusions, pins, bumps, and others) protuberance on at least a first elastic member which is captured by a receptacle of rigid or elastic second member (e.g., within holes, slots, ratchets, recesses, or other receptacles) to form a pivot point between the first and second implant members. The hinge can be prepared by the tab of a first implant member being coupled or otherwise received into the receptacle of a body of an adjacent implant member. When the tab of a first implant member is received into the receptacle of an adjacent second implant member, the first and second implant members become pivotally locked together. When unlocked, as when the tab of the first implant member is not received into the receptacle of the adjacent second member, the first and second implant members can move freely with respect to each other. In one instance, the unlocked first and second implant members are not coupled in any manner. In other instances, the first and second implant members can be coupled together or otherwise associated (e.g., via a guidewire or hinge pin) but capable of moving or bending with respect to each other. Interlocking (e.g., locking and unlocking) can occur when the first and second implant members (e.g., tab and receptacles together) are compressed, tensioned, and/or rotated with respect to each other.

In one embodiment, the present invention relates to implants having two or more separate implant bodies that are coupled together with puzzle-shaped coupling members with a male coupling member on one body and a female coupling member on the other body. The coupled implant bodies can have both male and female puzzle coupling members that correspond in location to such that coupling the puzzle coupling members provides the functional implant. One or more of the implant bodies can include a puzzle surface with a puzzle protrusion that has some flexibility, where the puzzle protrusion can be a tab or ridge having a puzzle protrusion cross-sectional profile. The puzzle protrusion cross-sectional profile can include a tab that has a narrow neck and wider head, where the neck extends from the implant body with the head free to be received into a corresponding receptacle. The tab configuration can be shaped similar to a shaft with an enlarged head, while the ridge configuration can have a substantially consistent cross-sectional profile extending along the ridge. The tabs and ridges can be flexible so as to move a head thereof relative to a puzzle receptacle, which can be a hole recess or long narrow recess similar to a slot or groove, with a narrower opening than cavity cross-sectional profile. The cross-sectional profiles of the male and female puzzle members can match so that the male member can be received into and contained within the female member. The corresponding shapes can interlock with a male member cross-sectional profile that expands moving away from the surface of the puzzle surface before narrowing to a top that fits within a recess of the female member having a corresponding cross-sectional profile.

An embodiment of the invention includes an implant device configured to improve the functioning of an intervertebral disc positioned between, contacting, and/or separating a pair of vertebrae in a spine having a longitudinal axis. The implant can be implanted between the vertebrae in order to increase a height (H) of the space or disc between the vertebrae and thereby to decrease the width (W) of the disc. The implant device can be configured to selectively contact at least one vertebral surface of the pair of vertebrae and to separate the pair of vertebrae along the longitudinal axis of the spine. This can increase the height (H) of the disc or disc space, and decrease perpendicularly the width (W) of the damaged disc to reduce pressure on nerves adjacent said disc.

In one embodiment, the present invention includes a multi-member implant that has multiple implant members coupled together through locking tabs that are received into locking receptacles. The tabs are configured with a stem and free head, where the stem is narrower or smaller than the head, and the receptacles have corresponding shapes. That is, the receptacles have narrower or smaller openings and larger cavities, where the narrow opening receives the stem and the larger cavity receives the head. The implant can be configured as a multi-membered sandwich or train.

In one embodiment, the implant includes a plurality (e.g., two or three or more) of separate implant members that are stacked in a sandwich format. The implant members may be planar or volumetric, where stacking the implant members expands or increases the height of the implant. In one example, the implant can include a top member, center member, and bottom member. The top member and bottom member may be separate or may be two opposing sides of a clamshell configuration that receives the center member into the open clamshell. The multi-member implant can be formed when the center member is inserted between the top and bottom members. The clamshell can include the top and bottom members pivotally coupled at an end. The clamshell can include chamfered ends on the ends of the members that pivot away from each other when the clamshell is opened. When the center member enters the chamfered end of the clamshell, the top and bottom members pivot in opposite directions. The center member can be inserted into the clamshell before, during, or after implantation between adjacent vertebrae. In the clamshell configuration, the center member spreads the top and bottom portions in either a linear or arcuate direction.

In one embodiment, the clamshell member and center member can include cooperative notches and teeth that fit in the notches, which can allow for reversibly interlocking the center member with the clamshell member. The cooperative notches and teeth can provide a ratchet-type interlocking as the center member is inserted into the clamshell. The center member may be inserted into the clamshell so as to retain the clamshell in an open or expanded orientation, or so that the clamshell closes onto the center member in a closed orientation. The pivot end of the clamshell may also be decouplable or have a tab and receptacle configuration so that the top and bottom members can detach from each other. As such, the center member can be received into the clamshell while the clamshell is capable of pivoting, or the top and bottom members can be separated and recoupled with opposite sides of the center member, either of which can couple the center member with the top and bottom members by a reversible locking mechanism.

In one embodiment, the multi-membered implant can be assembled in vivo or within the disc space between adjacent vertebrae. For example, the multiple members can be “snap-fit” with locking tabs being received into locking receptacles.

As described, the multi-membered implant can be assembled prior to insertion, when partially inserted, or after being fully inserted into a disc space between adjacent vertebrae. The implant can be assembled by moving the separate members in any direction that is allowed in order for a tab to be received into a receptacle. This can include snap-fit pressing the tab into the receptacle, or sliding the tab into a groove receptacle from on open end. It can be preferable that at least one implant member is more elastic; however, the different members can be of the same material or elasticity.

The elasticity of at least one tab or body defining a receptacle enables the tab to enter the narrower portion of a recess with less force or lower pressure or lower stress. The elasticity of the tab allows the tab to expand within the wider portion of the receptacle. Alternatively, the elasticity of the body that defines the receptacle allows for the receptacle to expand to receive the tab therein.

The engaging surfaces can include one or more omega recesses (“Ω”). The omega recesses can be short (e.g., hole-like) or elongate (e.g., trough-like), and may extend partially or all the way across the engaging surfaces. The omega recesses can allow for bone ingrowth and interlocking of the engaging surfaces and vertebrae. The omega recesses allow for the engaging surfaces to engage with the vertebrae so as to be in fixed locations.

The implant can be used to manipulate and revitalize a spinal column disc while minimizing or preventing the removal of material comprising the disc. The implant can be inserted in the disc either through a pre-existing rupture or through an opening formed in the front, back, or sides of the disc. Increasing the space between the vertebrae bounding the disc or removing disc material often is not necessary to insert the implant device in the disc. The implant device generates internal traction or other forces acting on the disc to alter the shape of the disc. The shape of the disc is altered to relieve pressure on nerves adjacent the disc. The shape of the disc is also altered to draw nuclear hernias back into the interior of the disc and to produce a disc shape that improves functioning of the disc.

In one embodiment, an implant can include a first member having a tab with one or more protuberances, and a second member having one or more receptacles configured to receive the tab having the one or more protuberances. The one or more protuberances and protuberance-receiving receptacles can be cooperatively configured to be coupled so as to form a joint between the first and second members. The coupling can be removable so that the first and second members can be decoupled. The coupling can be permanent so as to fix the first and second members together. The tab can be configured to be reversibly deformable or bendable or pivotal. The tab can have an elastic member that allows for articulation of the one or more protuberances, where the tab can have a stem that is elastic and deformable or bendable or pivotal. The tab can have an elastic head that can be deformed or pressed in so as to be capable of being received through a narrow opening in the receptacle before expanding in a larger cavity of the receptacle such that the tab interlocks with the receptacle. In one aspect, the body defining the protuberance-receiving receptacles can be elastic so as to open or stretch apart to receive the tab and then to elastically close or retract together around the tab so as to interlock the tab within the receptacle. In one aspect, the one or more tabs and/or tab protuberances can be configured with a slit that allows the one or more tabs and/or tab protuberances to narrow or contract when inserted into the corresponding receptacle. In one aspect, the one or more tabs and/or tab protuberances can include a pin (e.g., spherical pin) shaped and dimensioned to be received into a groove or slot in the one or more receptacles. In one embodiment, the one or more tabs and/or protuberances and corresponding receptacles can have cooperating ratchet members so that the tabs ratchet with respect to the receptacles.

In one embodiment, the implant can be configured to be expanded in an axis so as to separate the adjacent vertebrae. The implant can be expanded like a clamshell or as described in the incorporated references, such as having a rotatable cam that selectively exposes engaging surfaces. In one aspect, the implant can be locked in an expanded configuration. The implant can have an expanded configuration that is capable of moving in one or more directions. The implant can have an expanded configuration that is locked in position so that it does not move in any direction.

The implant can include one or two engaging surfaces configured to engage surfaces of the adjacent vertebrae. The engaging surfaces can include one or more engaging features, which can be protrusions that insert into tissue or bone, or which can be tissue or bone-receiving recesses with a wider recess cavity and narrow recess opening. These tissue- or bone-receiving recesses can be omega shaped.

In one embodiment, the implant can have a center member, a top member, and a bottom member. The top member and/or bottom member may be configured with a harder material for being attached to and integrating with the bone. The center member can be a softer material with higher elasticity, which higher elasticity can allow for coupling between tabs and receptacles as described herein. The softer material can be of a lighter weight with less density, which can improve the implant. The softer material may also provide some shock-absorbing capability between the two adjacent vertebrae. For example, the top and bottom members can be metal, such as stainless steel, and the center member can be a polymer, such as a polyether ether ketone (PEEK). However, the different members may be of any different materials to take advantage of the different properties of the different materials. As such, it may be suitable for the top and bottom members to be more elastic with the center member being harder. Also, the PEEK member can be radiotranslucent while the metal can be radiopaque. The metal members can be any of various metals, such as titanium or tantalum.

In one embodiment, the top member and bottom member may be coupled through a pivot point so as together form a clamshell. The clamshell can open to a “V” shape and then snap-fit with the center member to form the sandwich format implant. One advantage of the clamshell is that the height dimension increases as the clamshell opens, and the angle of the open clamshell can increase because the center member functions as a wedge. Alternatively, the top and bottom members can decouple at the pivot point so that front and back of the top and bottom members open symmetrically. Alternatively, the top and bottom members can be separate that snap-fit with the center member to form the sandwich format implant. Accordingly, the top and bottom members can be configured for linearly expanding or arcuately expanding, and either be fixed in the expanded orientation or snapping down onto the center member for a sandwich format.

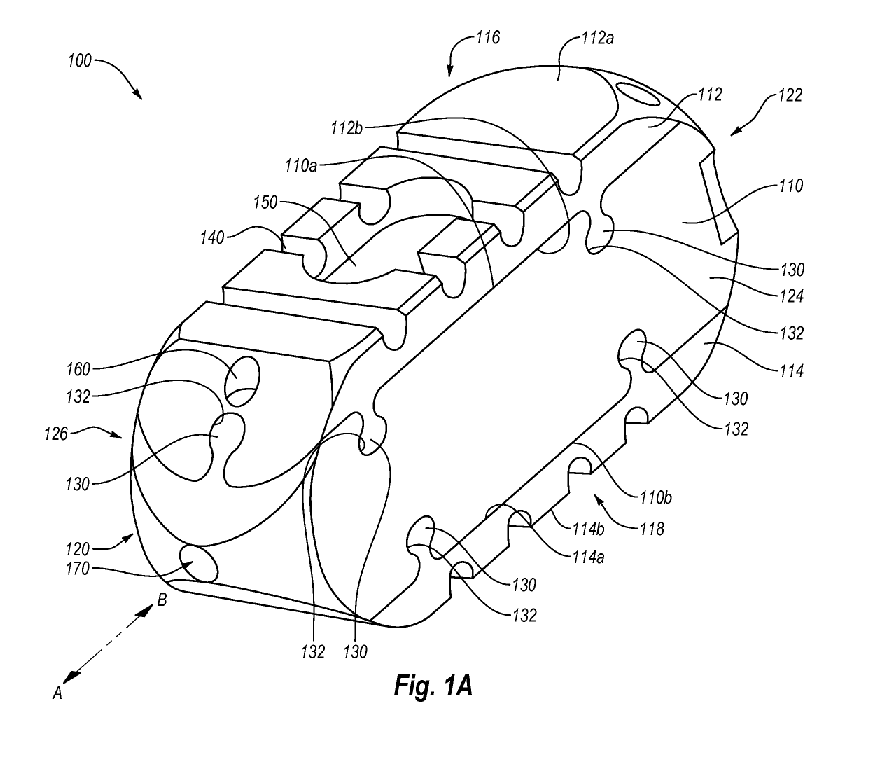

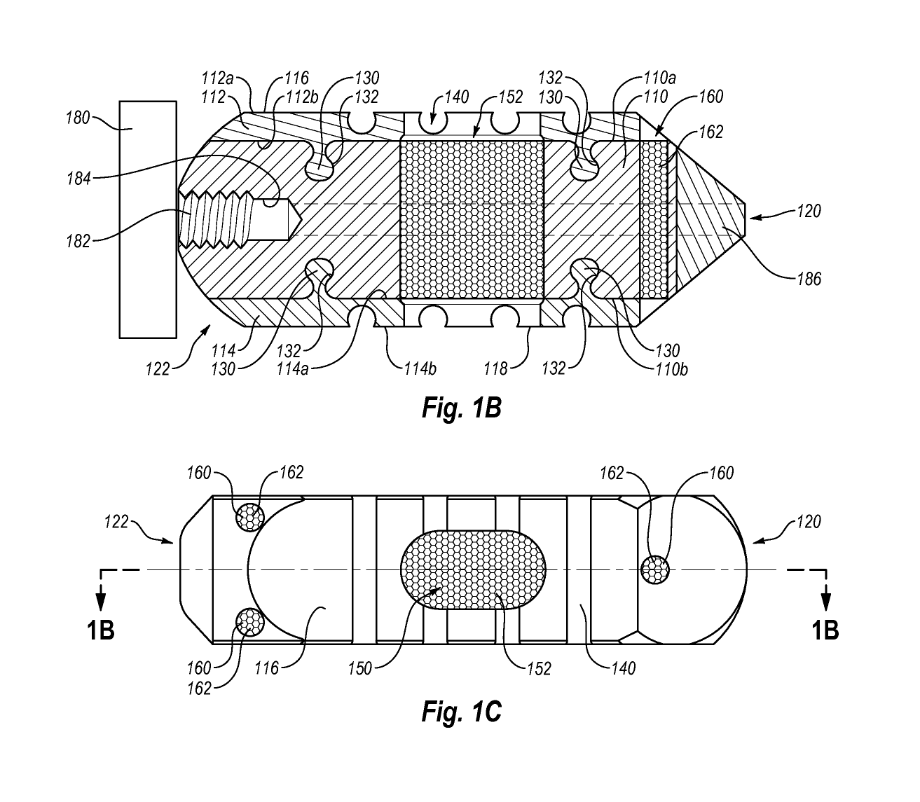

FIGS. 1A-1C illustrate a multi-membered implant 100 having a center member 110 between a top member 112 and a bottom member 114. The center member 110 has a top side 110a coupled to a bottom side 112b of the top member 112, and has a bottom side 110b coupled to a top side 114a of the bottom member 114. The top member 112 has a top side 112a opposite of a bottom side 114b of the bottom member 114. The implant 100, and thereby the center member 110, top member 112, and bottom member 114, can include a top side 116, bottom side 118, front end 120 and a back end 122, a first side 124, and a second side 126. For example, the front end 120 can be tapered or have a slope from the top side 116 and/or bottom side 118 to a point, which point can include a longitudinal conduit 170 that extends to the back end 122. The longitudinal conduit 170 can be configured as a guidewire conduit, and while it is shown on a center axis, it can be lateral towards the top side 116, bottom side 118, first side 124, or second side 126. The back end 122 is shown to be rounded. However, the implant 100 can have other shapes.

The top side 116 and/or bottom side 118 can be configured as engaging surfaces, which can include one or more engaging features that engage with the vertebrae or disc tissue. As shown, the engaging features can include omega recesses 140, which are illustrated as grooves extending from the first side 124 to the second side 126. However, the omega recesses 140 can be short or hole-like and may be at any angle as well as extending from the front end 120 to the back end 122.

The top side 116 is shown to include a shuttle recess 150 that is dimensioned to receive cargo therein. Such cargo can be filler material that is porous (e.g., bone or metal with pores), and may include active agents, such as growth factor, to promote bone growth. While not specifically shown, a shuttle recess 150 can be included on the bottom side 118, first side 124, and/or second side 126. The shuttle recess 150 can extend from the top side 116 through the implant 100 to the bottom side 118, or partially therethrough, such as only being in the top member 112 and/or bottom member 114. However, a filler member 152 (e.g., filler material) can be located therein.

The implant 100 is also shown to include at least one lateral conduit 160 extending from the top side 116 to the bottom side 118. The lateral conduit 160 is optionally filled with a filler member 162, which can be the same or different from the filler member 152, and may include the porous material. In one example, the filler member 162 can be radiopaque.

The center member 110, top member 112, and/or bottom member 114 can include tabs 130 and corresponding tab receptacles 132 so that these members can be coupled together into the illustrated implant 100. The tabs 130 are shown to have a narrow stem and larger head, where the cross-sectional profile of the stem is smaller than the cross-sectional profile of the head. The receptacles 132 are shown to have a narrower opening and larger internal cavity, where the cross-sectional profile of the opening is smaller than the cross-sectional profile of the cavity. The shapes and/or dimensions of the stem of the tab 130 and opening of the receptacle 132 can match or be about the same so that the stem fits into the opening. The shapes and/or dimensions of the head of the tab 130 and cavity of the receptacle 132 can match or be about the same so that the head fits into the cavity. This configuration allows for the tab 130 to interlock with the receptacle 132, and can be considered to be similar to a jigsaw puzzle interlock.

FIG. 1A shows the top member 112 to include a receptacle 132 on the bottom side 112b near the front end 120 and a tab 130 near the back end 122, and shows the bottom member 114 to include a tab 130 on the top side 114a near the front end 120 and a receptacle 132 near the back end 122. Correspondingly, the center member 110 includes a tab 130 on the top side 110a near the front end 120 that matches with the receptacle 132 of the top member 112, and a receptacle 132 near the back end 122 that matches with the tab 130 of the top member 112. Also, the center member 110 includes a receptacle 132 on the bottom side 110b near the front end 120 that matches with the tab 130 of the bottom member 114 and a tab 130 near the back end 122 that matches with the receptacle 132 of the bottom member 114. These tabs 130 are generally laterally oriented. FIG. 1A also shows a front tab 130 that is perpendicular with the other tabs 130, and extends from the front end 120 toward the back end 122.

FIG. 1B shows the top member 112 and bottom member 114 having tabs 130 that are received into receptacles 132 of the center member 110. However, any combination of tabs 130 and corresponding receptacles 132 can be included on any of the center member 110, top member 112, and/or bottom member 114 in order to couple these members together into the implant 100. The implant 100 can be formed by snapping these members together by pressing the adjacent surfaces together, which presses the tabs 130 into the receptacles 132. Alternatively, the tabs 130 can be ridges and the receptacles 132 can be slots, and the members can be slid laterally with respect to each other, such as by inserting the first side 124 of a tab 130 into a second side 126 of a receptacle 132 and sliding the members with respect to each other. The filler member 152 and/or 162 can be then inserted to fix the members 110, 112, and 114 with respect to each other and to inhibit further relative sliding.

FIG. 1B shows a cross-sectional profile which illustrates the filler members 152 and 162. Also shown is an optional endplate 180, which can be fixed or rotational, and may have fastener apertures as described herein and in the incorporated references. The endplate 180 is shown to be attached to a threaded shaft 182 that is received into a threaded recess 184; however, the endplate 180 can be integrated with the center member 110 or be coupled to a shaft or cam mechanism. Additionally, an internal lumen 186 is shown to extend from the front end 120 to the back end 122, which can be the longitudinal conduit 170, and be configured for receiving a guidewire or it can be configured to receive a shaft of a cam mechanism. When a cam mechanism is included, the filler member 152 can be configured as a cam mechanism that can selectively extend an engaging surface through the shuttle recess 150 as described in the incorporated references.

The shuttle recess 150 and/or lateral conduit 160 can independently extend at least partially through, or completely through the implant 100. When filled, the shuttle recess 150 and/or lateral conduit 160 can function to maintain the position of the different members 110, 112, and 114 of the implant 100. The shuttle recess 150 and/or lateral conduit 160 can be empty or partially or completely filled with a material, such as filler members 152 and 162, respectively. The filler members 152 and/or 162 can be bone graft or other porous materials, such as porous metals. With materials of different densities, the filler members 152 and/or 162 can function as a marker to identify the position of implant 100 in a patient's body. The filler members 152 and/or 162 can be held by a lip (e.g., tab or any protuberance) positioned anywhere along shuttle recess 150 and/or lateral conduit 160, or shuttle recess 150 and/or lateral conduit 160 can have smooth surfaces without a lip when filler members 152 and/or 162 are press-fit into shuttle recess 150 and/or lateral conduit 160.

FIG. 1C includes a top view of the implant 100, which shows two lateral conduits 160 having filler members 162 at the back end 122.

In one embodiment, the implant 100 can be assembled by pressing the center member 110 into the top member 112 and bottom member 114 in the direction normal to the direction of arrows A and B (illustrating the longitudinal direction). The tabs 130 are shaped as puzzle tabs that can interlock with puzzle receptacles 132 in order to interlock center member 110 with top member 112 and/or bottom member 114. The front tab 130 and front receptacle 132 can be used to assemble the implant 100 when a force is applied in the direction of arrows A and B, which can be threaded, smooth, toothed, ratcheted, or the like.

In one embodiment, the center member 110 can be constructed of a polymer and the top member 112 and bottom member 114 can be constructed of metal, such as porous metal. The top side 116 and/or bottom side 118 can include surfaces that have omega recesses and/or are porous for bone ingrowth from the adjacent vertebral bodies. In one aspect, the center member 110 can be elastic with a modulus of elasticity similar to bone. When the different members are of different materials or different densities (e.g., different porosities) the members with the greater density can function as radiopaque markers to identify the implant 100 in a patient's body (e.g., human or animal) using medical imaging.

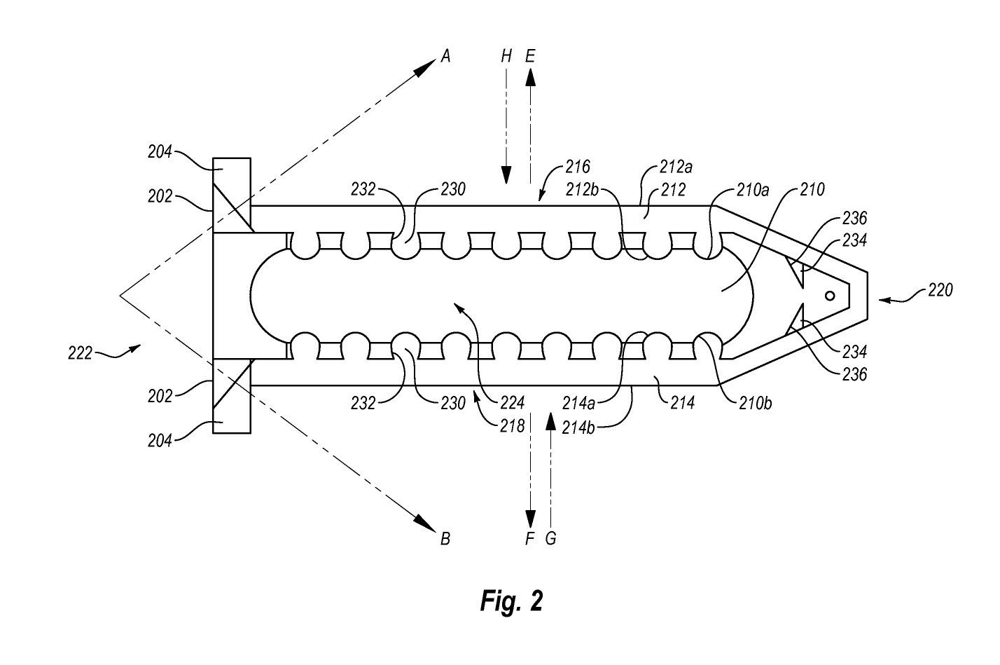

FIG. 2 illustrates an embodiment of a multi-membered implant 200 in a clamshell format having a center member 210 between a top member 212 and a bottom member 214. The center member 210 has a top side 210a coupled to a bottom side 212b of the top member 212, and has a bottom side 210b coupled to a top side 214a of the bottom member 214. The top member 212 has a top side 212a opposite of a bottom side 214b of the bottom member 214. The implant 200, and thereby the center member 210, top member 212, and bottom member 214, can include a top side 216, bottom side 218, front end 220 and a back end 222, a first side 224, and a second side (not shown). For example, the front end 220 can be tapered or have a slope from the top side 216 and/or bottom side 218 to a point, which point can include a longitudinal conduit (not shown) that extends to the back end 222 configured as described herein. The longitudinal conduit can be configured as a guidewire conduit, and while it is shown on a center axis, it can be lateral towards the top side 216, bottom side 218, first side 224, or second side 226. The back end 222 is shown to include an endplate 204 having fastener apertures 202 configured for receiving fasteners therethrough, such as bone screws. The implant 200 is configured in a clamshell format with the top member 212 and bottom member 214 forming a clamshell through the front end, which serves as a pivot point for opening and closing of the clamshell. While not shown, the top side 216 and bottom side 218 may have engaging surfaces configured to engage with the vertebra or disc material therebetween, and the engaging surfaces (e.g., 116 and 118) can include at least one omega-shaped recess or groove for bone ingrowth and interlocking the vertebra and implant 200.

The top side 210a and bottom side 210b of the center member 210 are shown to have a series of omega recesses 232 and the bottom side 212b of the top member 212 and top side 214a of the bottom member 214 are shown to have a corresponding series of omega-shaped tabs 230 received into the omega receptacles 232. The omega tabs 230 are shown to have a short stem with a large omega-shaped head; however, the stem could be extended any distance. The center member 210 is shown to narrow at the front end 220 by the top side 216 and bottom side 218 tapering to the front end 220. This portion can include one or more notches 234 on one or both sides which receive teeth 236 of the top member 212 and bottom member 214 or the front end 220 portion of the clamshell where they are shown. The clamshell format allows for the center member 210 to be inserted into the clamshell until reaching the front end 220 of the clamshell so that the top member 212 and bottom member 214 can be closed onto the center member 210 for coupling and forming the multi-member implant 200. For example, the top member 212 can be pivoted in the direction of arrow E and the bottom member 214 can be pivoted in the direction of arrow F in order to receive the center member 210, and then pivot in the direction of arrows G and H to close down onto and couple with the center member 210.

In an alternative of FIG. 2, the top member 212 and bottom member 214 can be disconnected or otherwise separated and configured to sandwich the center member 210.

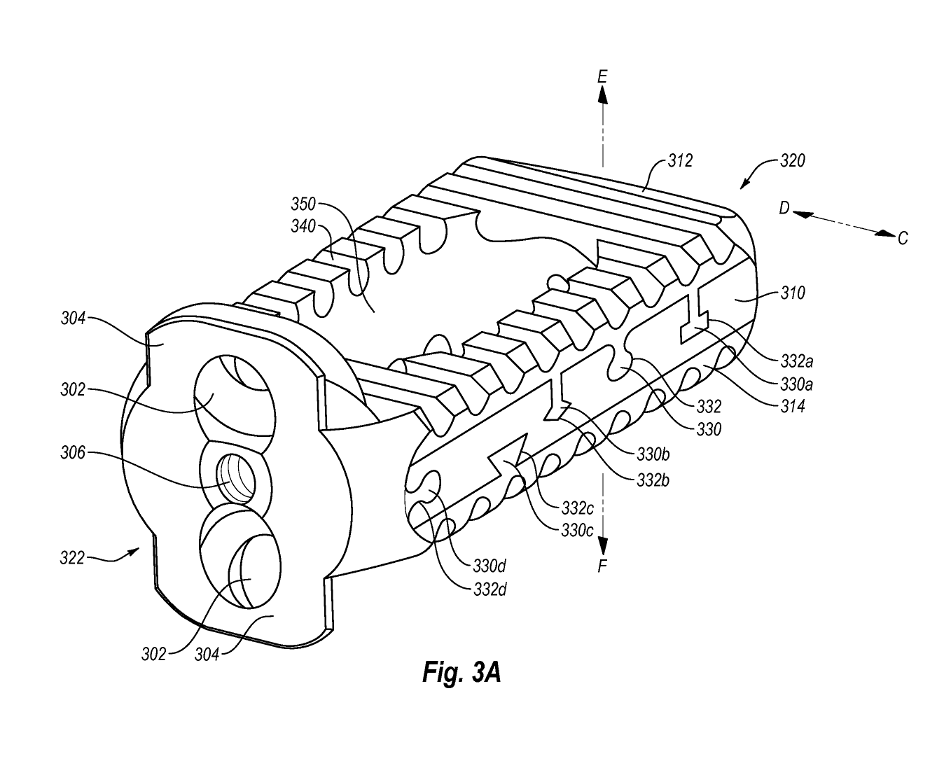

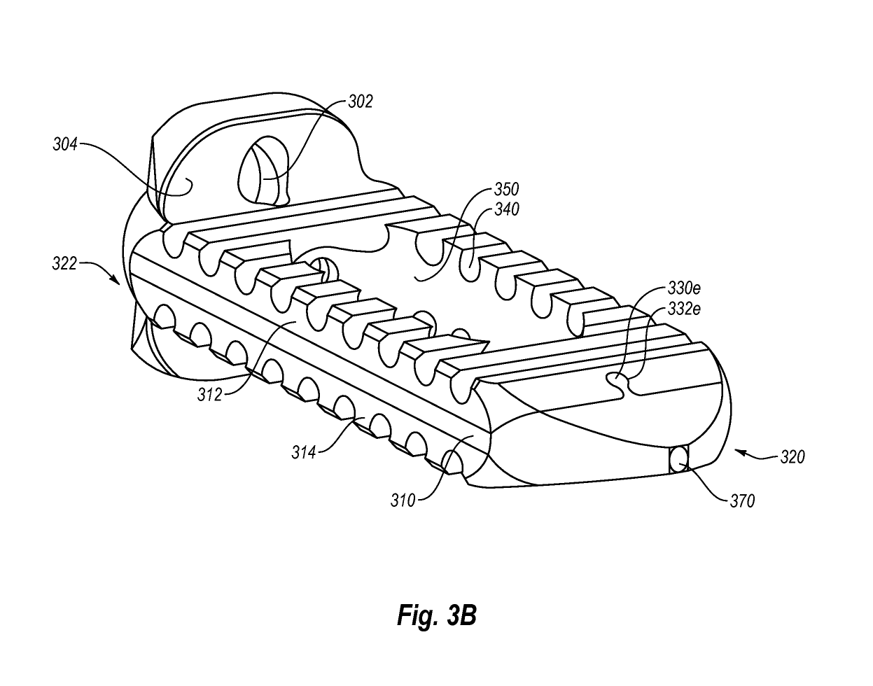

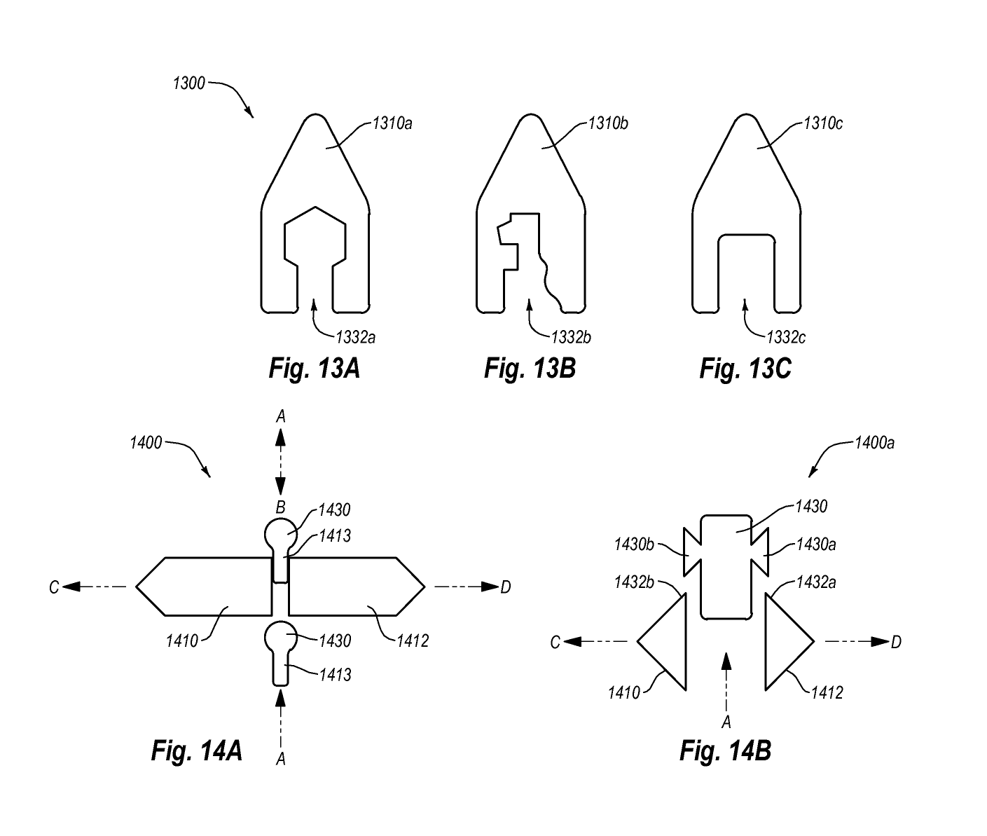

FIGS. 3A-3B show another embodiment of the multi-membered implant 300 having a center member 310 that is coupled to a top member 312 and a bottom member 314. The back end 322 of the implant 300 is shown to have an endplate 304 having bone fastener apertures 302, and having a center aperture 306 that is threaded. The center aperture 306 can be a conduit that extends to the front end 320. The top member 312 and bottom member 314 both are shown to have vertebra-facing surfaces with omega recesses for bone ingrowth, and a shuttle recess 350. Here, the implant 300 is shown to have the center member 310 have interlocking receptacles 332 that are configured to receive and interlock with interlocking tabs 330 of the top member 312 and bottom member 314. The interlocking tabs 330 and receptacles 332 can have various interlocking shapes where the tab has a larger head than stem, as shown by exemplary shapes of 330a,332a, 330b,332b, and 330c,332c; however, any interlocking shape can be used (see FIG. 13). Also, the center member 310 can have interlocking tabs and the top member 312 and bottom member 314 can have interlocking receptacles.

FIG. 3A shows the back end 322 of the center member 310 to include an interlocking receptacle 332d received into an interlocking tab 330d of the back end 322. As such, the back end 322 can be the pivot point of a clamshell format with the top member 312 and bottom member 314.

Alternatively, the back end 322 and front end 320 can be members that are coupled or integrated with the top member 312 and bottom member 314 so as to form a clamshell having a center configured and dimensioned to receive the center member 310 by inserting the center member 310 into the center of the clamshell and sliding the center member 310 along tabs 330 in the form of rails from one side of the implant 300 to the other side. Here, the shuttle recess 350 can extend from the top side to bottom side and be filled with a filler member of a filler material, such as a porous metal or bone so that vertebral bone grows into it. The filler member and subsequent bone ingrowth can lock the center member 310 in position within the clamshell.

FIG. 3B shows the top member 312 and bottom member 314 that are linked through the back end 322 to form a U-shaped slot that can receive the center member 310 as shown. Here, the implant 300 is shown with a top member 312 having an interlocking receptacle 332e extending from the front end 320 along a longitudinal direction, and which may extend all the way to the shuttle recess 350. The bottom member 314 may also have a similar interlocking receptacle. The center member 310 has an interlocking tab 330e that corresponds with the interlocking receptacle 332e, such that the center member 310 can be slid between the top member 312 and bottom member 314 in the longitudinal direction. More than one longitudinal tab 330e and receptacle 332e can be included. While not shown, the facing surfaces of the top member 312 and bottom member 314 can have lateral interlocking tabs and/or recesses for coupling with corresponding lateral interlocking tabs and/or recesses of the center member 310.

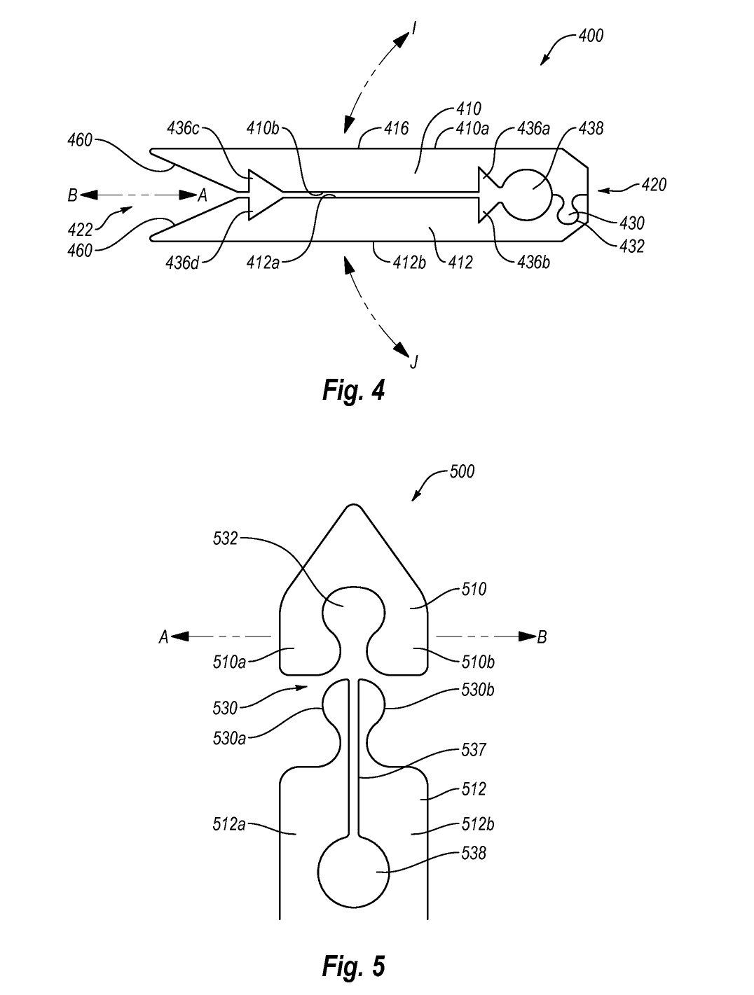

FIG. 4 shows a clamshell 400 having top member 410 and bottom member 412 in a clamshell format that can receive a center member (not shown) therebetween. The top member 410 includes a top surface 410a opposite of a bottom surface 412b of the bottom member 412, and with a bottom surface 410b of the top member 410 adjacent to the top surface 412a of the bottom member 412. The clamshell 400 has a front end 420, back end 422, top side 416, and bottom side 418. The back end 422 is shown with the top member 410 having a top chamfered end 460 and the bottom member 412 having a bottom chamfered end 462. The top chamfered end 460 can be adjacent to a top notch 436c and the bottom chamfered end 462 can be adjacent to a bottom notch 436d. The chamfered ends 460 and 462 cooperate to facilitate separation and pivoting of the chamfered ends 460 and 462 away from each other when receiving a center member, and the top notch 436c and bottom notch 436d can receive teeth of the center member, which can function as a temporary interlocked position during implantation. The facing surfaces (e.g., 410b and 412a) can rotate away from each other as shown by arrows I and J. The front end 420 of the implant flex aperture 438 that facilitates opening of the clamshell 400, and which implant flex aperture 438 is formed by shaping of the top member 410 and bottom member 412 appropriately. The front end 420 of the top member 410 can include an interlocking tab 430 that is received into an interlocking recess 432 of the bottom member 412, which flexes during opening of the clamshell 400. When the center member is being inserted, one or more notches 436a on the top member 410 and one or more notches 436b on the bottom member 412 can function as ratcheting fasteners so that the center member can ratchet the top member 410 and bottom member 412 pivotally away from each other. The front end 420, interlocking tab 430 and recess 432 can decouple from each other and sandwich onto the center member in some embodiments or stay interlocked after implantation. The principles of the clamshell 400 of FIG. 4 can be applied to the other implant embodiments described herein, such as in FIGS. 1A-1C, 2, and 3A-B, and features of those implants can be applied to the clamshell 400.

FIG. 5 shows an embodiment of an implant 500 having a first member 510 and a second member 512, where the first member 510 includes an interlocking receptacle 532 and the second member 512 includes a corresponding interlocking tab 530. While the implant 500 is shown in a train format where the first member 510 is a lead member with a pointed end and the second member 512 links linearly, the implant 500 may have these features in the sandwich or clamshell formats. The first member 510 has lateral sides 510a and 510b that define the interlocking receptacle 532, which lateral sides 510a and 510b can flex outwardly as shown by arrows A and B in order to receive interlocking tab 530 therein. Also, the second member 512 is shown with the interlocking tab 530 having a slit 537 that partitions the tab 530 into lateral tab members 530a and 530b, which can be pressed toward each other to narrow the tab 530 for insertion into the receptacle 532. The slit 537 can also extend into the body of the second member 512 in order to form lateral portions 512a and 512b, which also can be pressed toward each other to narrow the tab 530. The second member 512 can also include a flex aperture 538 at the base of the slit 537 to facilitate flexing and narrowing of the tab 530.

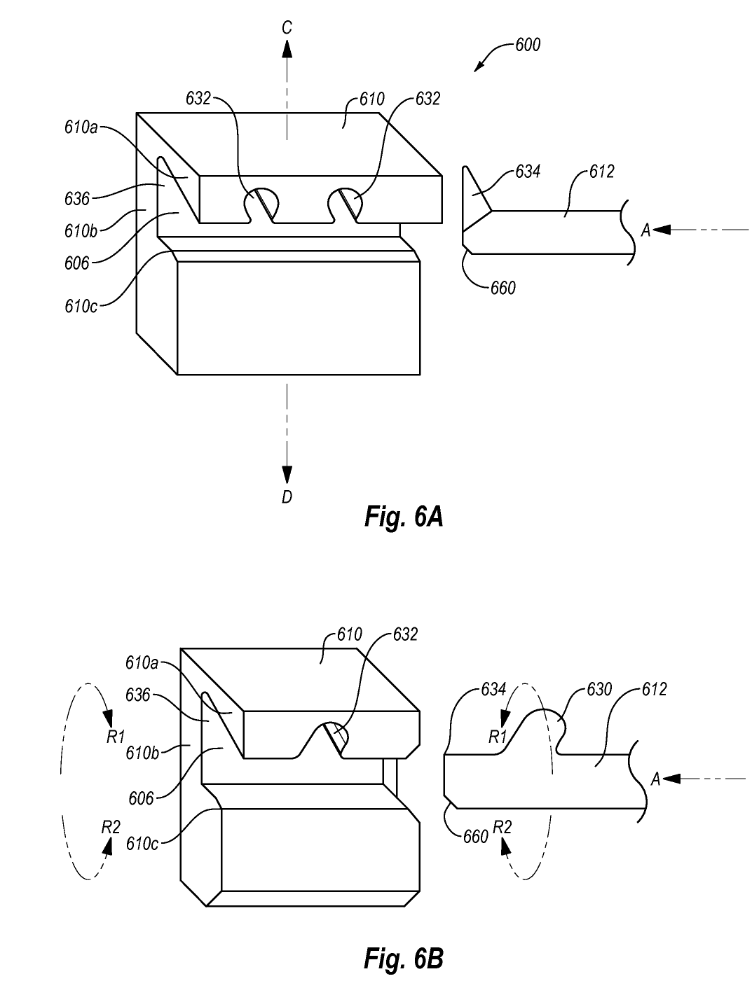

FIGS. 6A and 6B illustrate embodiments of interlocking configurations 600 between a first member 610 that is receiving a second member 612. Here, the first member 610 includes a first portion 610a, second portion 610b, and third portion 610c that cooperate to form a channel 607 that is dimensioned to receive the second member 612. The channel 607 may have one or more receptacles 632 for receiving one or more tabs 630 on the second member 612. The second member 612 can include a chamfered end 660 to facilitate insertion into the channel 607, which second member 612 may also include a tooth 634. The tooth 634 may be dimensioned and shaped to be received into a notch 636 of the first member 610, which notch 636 may be in the channel 607. While the channel 607 is shown to terminate at an end of the first member 610, the notch 636 and end can be internal of the first member 610. The first portion 610a and third portion 610c can flex away from each other in the direction of arrows C and D to facilitate insertion into the channel 607 in the direction of arrow A. In one aspect, the channel 607 can be U-shaped so that the tooth 634 can be oriented out the opening and optionally protruding therefrom, and after insertion the second member is rotated to slide the tooth 634 into the notch 636. In one option, the channel 607 can include an interlocking recess 632 that is selectively angled or offset and the second member 612 includes a similarly shaped interlocking tab 630. As shown, rotation in the direction of R1 locks the first member 610 and second member 612, and rotation in the direction of R2 unlocks these members. Optionally, the one or more sides of the opening of the channel 607 can be chamfered to facilitate insertion of the second member 612. Similarly, the second member 612 can include a chamfered end 660.

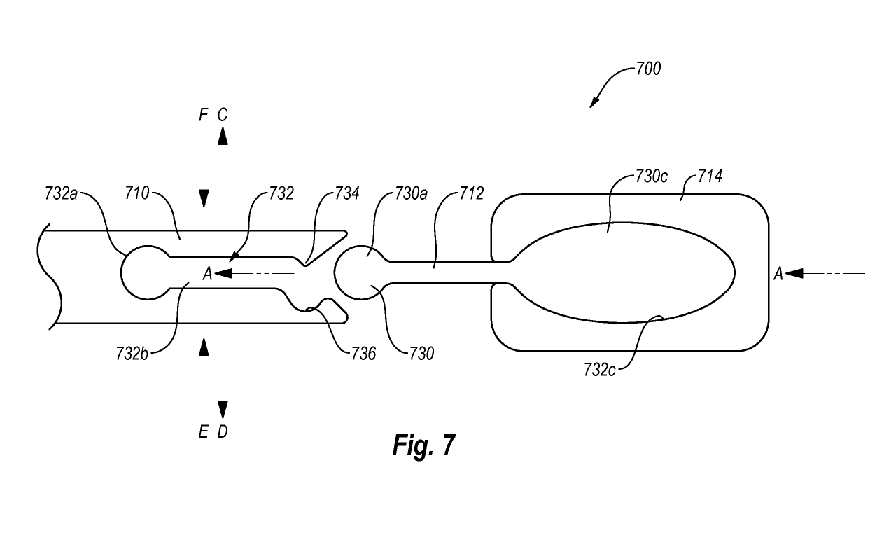

FIG. 7 illustrates an embodiment of an interlocking configuration 700 between a first member 710 and a second member 712. Also shown is the second member 712 interlocked with a third member 714, where this interlocking can be configured as shown with an elongated omega interlocking or it can be configured as the interlock between the first member 710 and the second member 712. The first member 710 is shown to include an interlocking receptacle 732 that has a notch 736 and tooth 734 on opposing sides near the opening that can be mated in a relaxed configuration or mated when pressed together. The receptacle 732 can include a large head portion 732a and a narrow stem portion 732b internal from the notch 736 and tooth 734. The second member 712 can include an interlocking tab 730 that includes a large head 730a that conforms with the large head portion 732a of the receptacle 732 and the tab 730 includes a narrow stem 730b that conforms with the narrow stem portion 732b of the receptacle 732. Insertion of the tab 730 into the receptacle 732 in the direction of arrow A flexes the sides in the direction of the arrows C and D until the head 730a is received into the head portion 732a of the receptacle 732, and then the sides can relax in the direction of arrows E and F to interlock the first member 710 and second member 712. While the interlocking configuration 700 is shown in a train format implant, it may be applied to a sandwich or clamshell format.

Also, the second member 712 can include a second tab 730c received into a receptacle 732c of the third member 714.

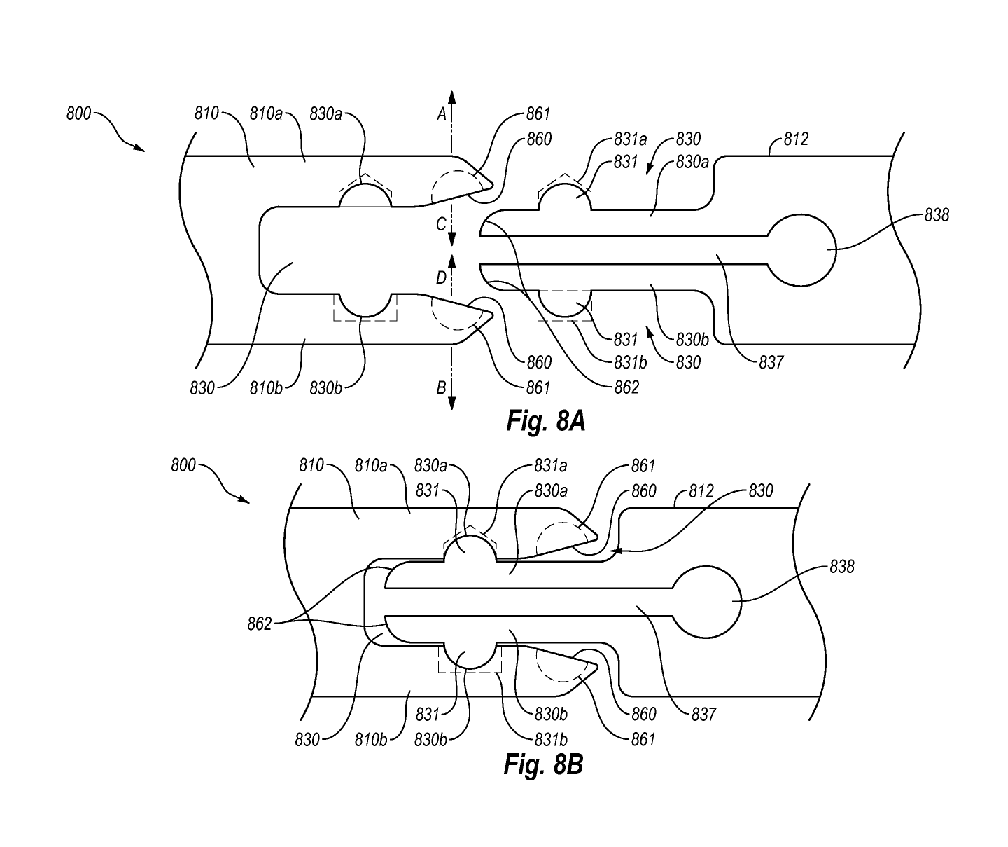

FIGS. 8A and 8B show an interlocking configuration 800 and method of interlocking that is similar to the interlocking configuration 700 of FIG. 700; however, the interlocking tab 830 of the second member 812 is shown to include retractable protrusions 831 (e.g., retractable balls or ball locks) that are received into recesses so that the retractable protrusions 831 can be spring activated with a spring in the bottom of the recess. However, the retractable protrusions 831 may be locked and fixed. While the protrusions 831 are shown to be spherical, they can be shaped as interlocking tabs or omega tabs as described herein. They may also be square or pointed as shown by the dashed lines (831a and 831B). While the protrusions 831 are shown to be intermediate, they can be at either end or position therebetween and any number can be included. The first member 810 can include corresponding receptacles 832a and 832b for receiving the protrusions 831. The tab 830 can also include lateral stem portions 830a and 830b that define an internal slit 837 that can be pressed together during coupling of the first member 810 to the second member 812 as shown in FIG. 8B. The slit 837 can also have a flex aperture 838 at its base. The first member 810 can have a first portion 810a that flexes in the direction of arrow A and a second portion 810b that flexes in the direction of arrow B during insertion of the tab 830, and then retracts in the direction of arrows C and D when the protrusions 831 are received into the receptacles 832a and 832b. Optionally, the opening of the receptacle 832 can include recesses 861 dimensioned to partially receive the protrusions 831 when insertion is initiated, and which can function to temporarily couple the first member 810 and second member 812. A series of recesses 861 could be used for a ratchet interlocking. In one option, the protrusions 831 can be placed on the tab 830 in a position that allows the first member 810 and second member 812 to pivot with respect to each other in the direction of arrows G and H.

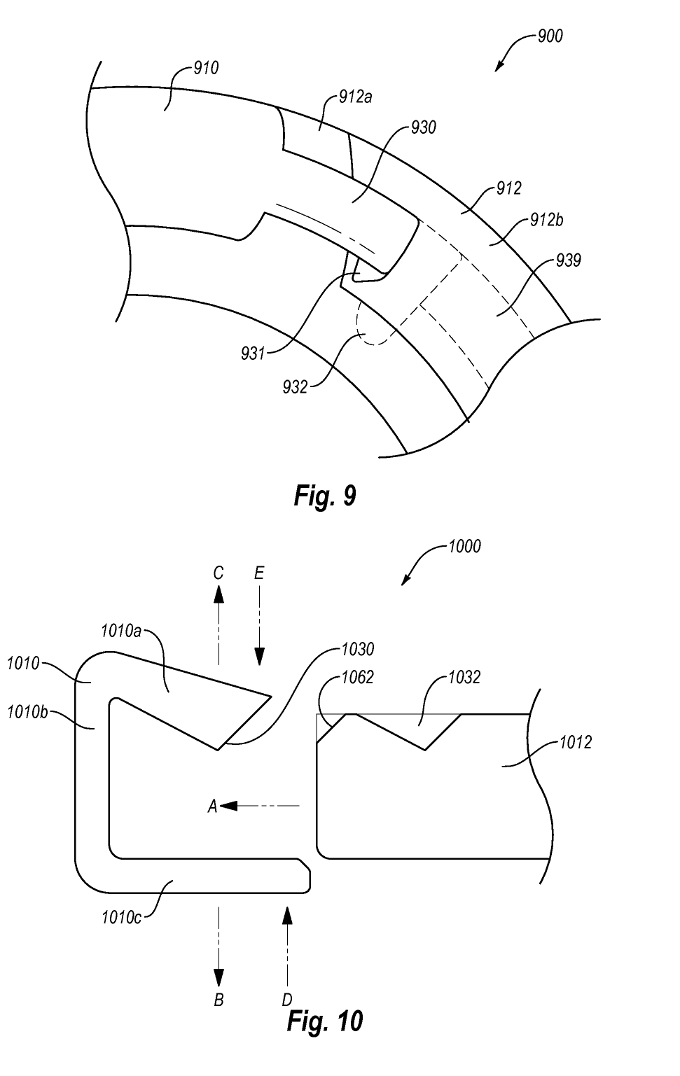

FIG. 9 illustrates an embodiment of a locking mechanism 900. Here, two or more different implant members can be linked together so that they may move with respect to each other, such as by rotating such as with a hinge or by flexible movement provided by a guidewire coupling the implant members. The first member 910 is shown to have a tab 930 that can slide along the second member 912, which sliding optionally can be along a groove 939 (dashed lines) over a first portion 912a and/or over a second portion 912b until a portion of the tab 930 is received into a receptacle 932. The tab 930 may have a tooth or other protuberance 931 that is received into the receptacle 932, which tooth 931 and receptacle 932 may be configured with corresponding interlocking shapes.

FIG. 10 illustrates an embodiment of a locking mechanism 1000 that removably locks a first member 1010 to second member 1012. The first member 1010 is shown to include a receptacle for receiving the second member 1012, which receptacle is defined by portions 1010a, 1010b, and 1010c. Portion 1010a can include a tab 1030, which here is illustrated as a triangle tooth, but which can have any tab or interlocking tab shape as described herein. The second member 1012 can include a chamfered end 1062 and a notch 1032 that together may optionally form a tooth protrusion as shown. When the second member 1012 is inserted into the first member 1010 in the direction of arrow A, the chamfered end 1062 pushes on the tab 1030 so that portion 1010a flexes in the direction of arrow C and the portion 1010c flexes in the direction of arrow B, and relaxes back in the directions of arrows D and E when the tab 1030 is received into the receptacle 1032.

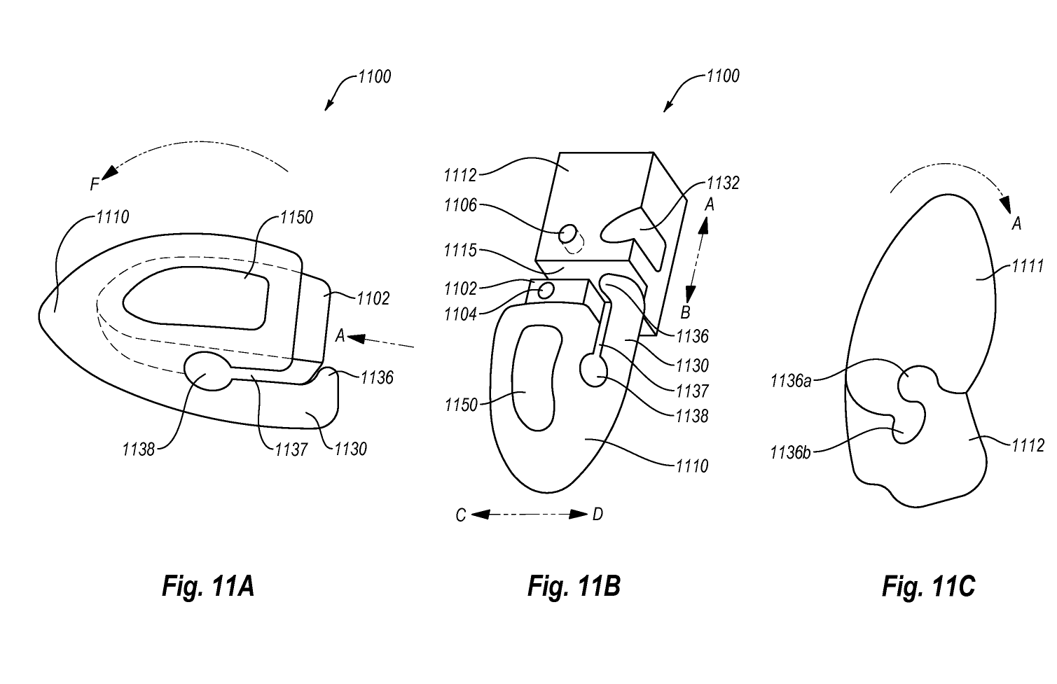

FIGS. 11A-11C show an embodiment of a modular implant 1100 having separate implant modules (e.g., 1110 and 1112) that couple together in a train format. A first implant module 1110 can be coupled to a second implant module 1112 by moving in the direction of arrow A, and uncoupled in the direction of arrow B. The first implant module 1110 is shown to include an interlocking tab 1130 that is shaped in conformation to an interlocking receptacle 1132 of the second implant module 1112. The tab 1130 has a protrusion 1136 at the end of its head or the head can have some other feature to be received into a corresponding receptacle 1132 for interlocking. The tab 1130 is shown to be adjacent to a slot 1137 with a flex recess 1138 at its base, which allows for increased flexibility and pivoting of the tab 1130. The first implant module 1110 can include a shelf 1102 that has a first fastener member 1104 that is fastened to a second fastener member 1106 of the second implant module 1112. The first implant module 1110 is shown to have a shuttle recess 1150, but the second implant module 1112 may also have one or more.

FIG. 11A shows that the first implant module 1110 and tab 1130 can be flexed with respect to each other as shown by the arrow F. The tab 1130 is configured as a locking arm that can have increased flexibility by the flexing aperture 1138 and slot 1137. FIG. 11B shows the first implant module 1110 coupling with the second implant module 1112, where the flexibility of the tab can allow for them to pivot with respect to each other, such as when the first fastener member 1104 and second fastener member 1106 are a pivot point, such as a pin in a hole or hinge. This allows relative flexibility shown by the arrows C and D. FIG. 11C shows another embodiment with the first member 1111 including the first tab 1136a and the second member 1112 including the second tab 1136b, which tabs 1136a and 1136b can be cooperatively shaped for interlocking with heads that fit adjacent to stems of the other.

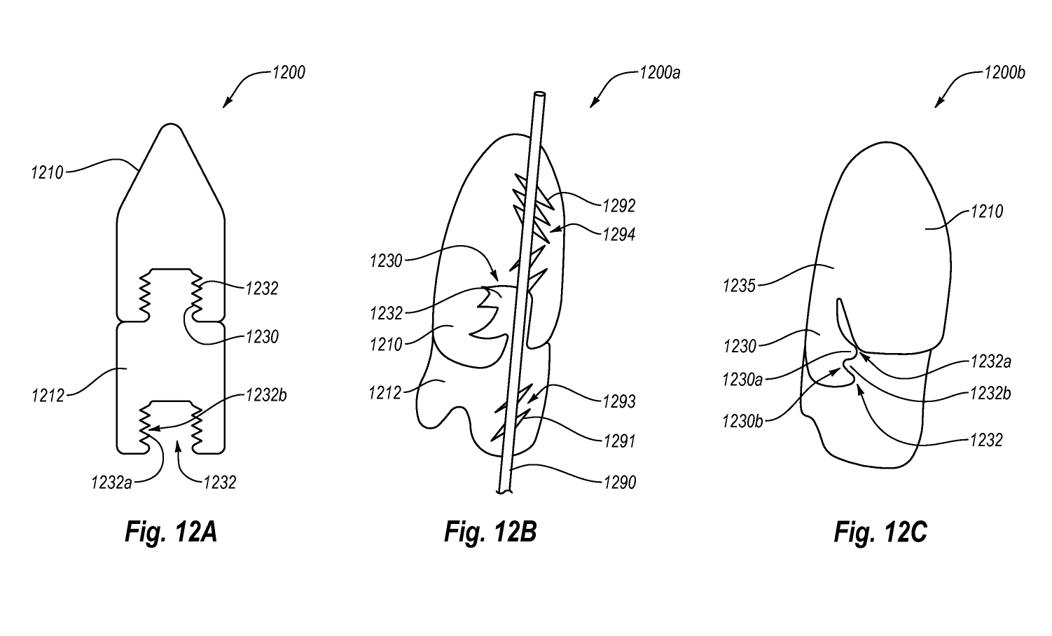

FIGS. 12A-12C show various configurations of ratchet interlocking mechanisms. As shown, the tabs can include ridges that form ratcheting members when the different implant modules slide relative to each other during interlocking. The ratchet interlocking mechanisms can ratchet when the first member 1210 is coupled with the second member 1212 by the interlocking receptacle 1232 receiving the interlocking tab 1230. The second member 1212 is shown to include a ratchet interlocking receptacle 1232 at the other end from the tab 1230. As can be seen in FIG. 12A, the surface can include ratchet protrusions 1232a and ratchet recesses 1232b that ratchet with corresponding features on the next implant module that is coupled thereto.

FIG. 12B shows an implant 1200a with a first member 1210 coupled to a second member 1212 through a ratcheted tab 1230 of the second member 1212 being received into a ratchet receptacle 1232 of the first member 1210. Also, the implant 1200a is shown to include a threaded wire 1290 (e.g., threaded guidewire or thin screw) that can have threads 1292 oriented in one direction with threads 1292 on the first member 1210, and have threads 1293 in the opposite direction with threads 1294 on the second member 1212, which upon rotation of the wire 1290 in one direction would pull the first member 1210 and second member 1212 together for coupling, and rotation in the opposite direction would push the first member 1210 away from the second member 1212 for decoupling.

FIG. 12C shows the implant 1200b having a first member 1210 with a ratchet tab 1230 that is connected via a pivot point 1235 that allows the tab 1230 to pivot with respect to the body.

FIGS. 13A-13C illustrate different interlocking mechanisms 1300, which shows a member 1310a having a polygon-shaped receptacle 1332a, a member 1310b having a key-shaped receptacle 1332b, and a member 1010c having a friction fit receptacle 1332c that is configured to friction couple with a corresponding tab.

FIGS. 14A-14B show a first locking mechanism 1400 having a first member 1410 being separated from a second member 1412 with a tab 1430. The tab 1430 is inserted therebetween in the direction of arrow A so that the neck 1412 separates the first member 1410 from the second member 1412. The head of the tab 1430 pressing against the first member 1410 and second member 1412 keep the tab 1430 from backing out, and functions as a locking mechanism. The second locking mechanism 1400a uses barbs, hooks, or other protrusions 1430a and 1430b that hook into the first member 1410a at a coupling point 1432b and into a second member 1412a at a coupling point 1432a. This can lock a member having the tab 1430 with one or two members having the first member 1410a and second member 1410b.

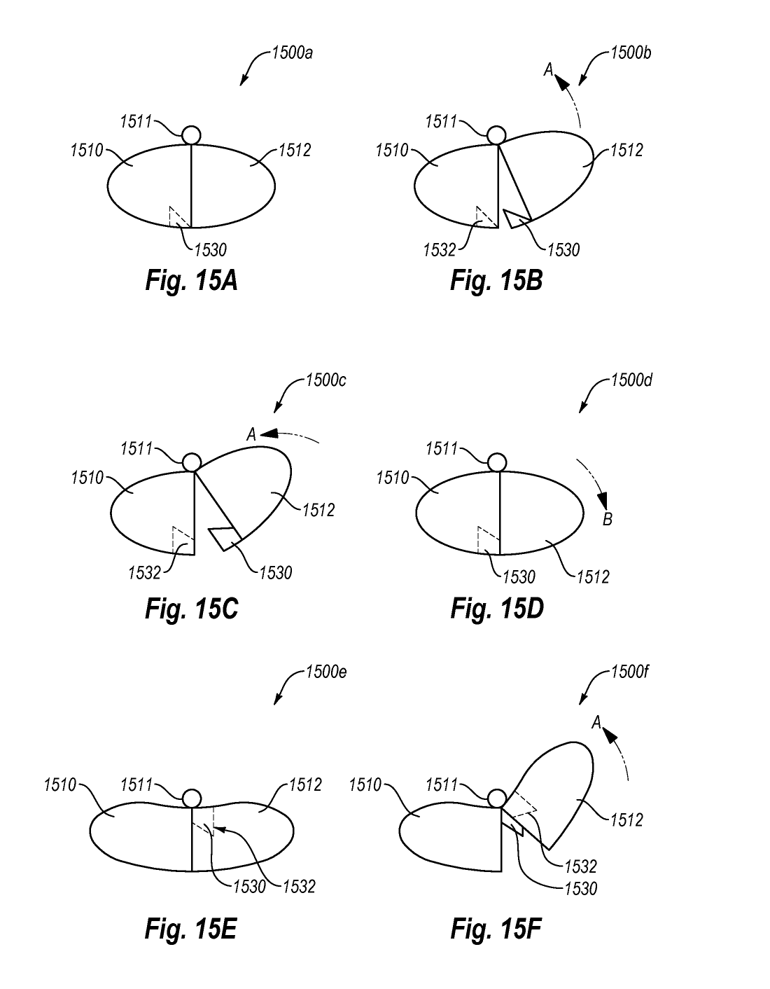

FIGS. 15A-15F show different embodiments of hinged interlocking mechanisms in the opened and closed positions. FIG. 15A shows a closed position of an implant 1500a having a first member 1510 coupled to a second member 1512 via a hinge 1511, where the first member 1510 has a receptacle 1532 that receives a tab 1530 of the second member 1512. FIG. 15B shows the open position of implant 1500b where once the tab 1530 is decoupled from the receptacle 1532, the tab is dimensioned or shaped to function as an interference tab that does not go back into the receptacle 1532, can rotate such as in the direction of arrow A. FIG. 15C shows the open position of implant 1500c by rotation in the direction of arrow A, which can be closed (FIG. 15D; implant 1500d) by the tab 1530 being received into the receptacle 1532 by rotation in the direction of arrow B. FIGS. 15A-15D show the tab and receptacle on the side that opens or that is opposite of the hinge; however, they can be positioned at any position on the facing surfaces. FIGS. 15E and 15F show the closed position (implant 1500e) and opened position (implant 1500f) with the tab 1530 and receptacle 1532 on the side with the hinge 1511.

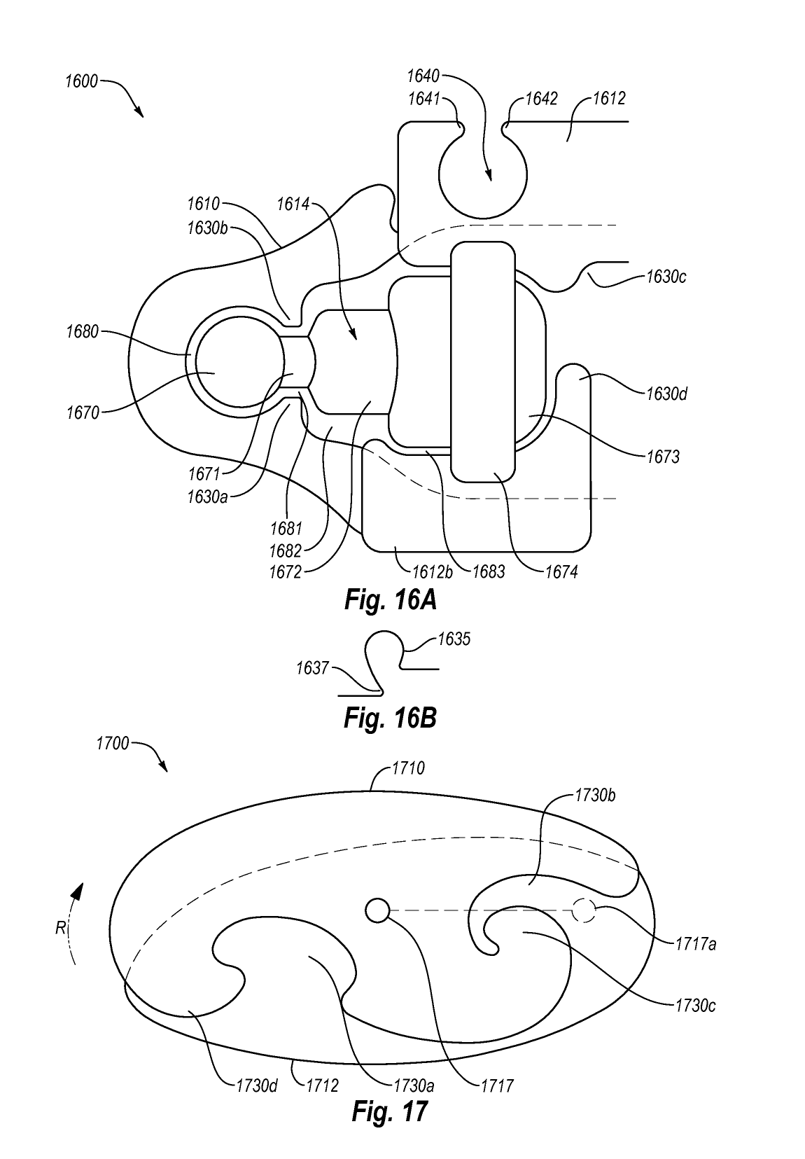

FIG. 16A illustrates an embodiment of a ball joint interlocking mechanism 1600, where a first member 1610 includes a receptacle 1632 that can function as a ball joint cavity for a tab 1630 of a second member 1612 to be a ball joint. The receptacle 1632 can include a head portion 1680 that has a head 1670 of the tab 1630 therein. The tab 1630 can include a stem 1671 adjacent to the head 1670 that is in a step portion 1681 of the receptacle 1680. The stem 1671 may protrude from a neck 1672 that is coupled to a tab body 1673 that has a cam 1674. The receptacle 1632 can include a neck portion 1682, body portion 1683, and a cam portion 1684. When the tab 1630 is rotated, the cam 1674 cams the cam portion 1684 so as to push member 1612a away from member 1612b. The first member 1610 can also include tabs 1630a and 1630b that are received by the neck 1672 of the tab 1630. The second member 1612 can include tabs 1630c and 1630d.

FIG. 16B shows an offset ratchet tooth with a top side 1635 and bottom side 1637. Also, FIG. 16A shows an offset omega recess 1640 with a top side 1641 and a bottom side 1642. These features can be employed with any of the implants described herein for coupling implant modules or for engaging disc tissue or vertebra.

FIG. 17 shows an embodiment of a cam mechanism 1700 that can be employed on any implant described herein or in the incorporated references. Here, the cam mechanism 1700 includes a first cam member 1710 and a second cam member 1712, which have cooperative tabs 1730a, 1730b, 1730c, and 1730d, that can interlock together as shown when the cam is closed. When the cam is opened, such as by rotation about pivot point or axis or axle 1717, the cooperative tabs 1730a, 1730b, 1730c, and 1730d unlock so that the first cam member 1710 rotates away from the second cam member 1712. The axle 1717 can be at select positions as shown by the dashed line and position of axle 1717a.

In one example, FIGS. 2, 3A, 3B, and 4 can illustrate assembly of an implant, which is configured with a top portion, a bottom portion, and center member intermediate therebetween. The top and bottom portions are shaped similar to a clamshell configuration and the center member is received therein. FIGS. 3A and 3B show perspective views of the implant. FIG. 4 shows a side view of the clamshell. The implant can be formed when the center member is inserted between intermediate portions of the clamshell in the direction of arrow A of FIG. 4. When the center member enters the chamfered end of the clamshell shown by arrow A, the center member moves the top and bottom portions in opposite directions of arrow I and arrow J. When the implant is inserted intermediate two vertebrae, and the center member rotates the top and bottom portions of the clamshell in opposite directions, the intervertebral disc and surrounding structures are compressed and/or tensioned, reshaping the disc. The center member can spread the top and bottom portions of the clamshell in either a linear or arcuate direction. Then as the center member moves in the direction of arrow A, the center member reversibly interlocks with the clamshell at the notches and corresponding protrusions.

The implant can be preassembled, or “snap-fit” in vivo. Tabs and recesses (i.e., notches) can have any configurations, such as configurations illustrated or as desired. These tabs and recesses can be shaped similar to puzzle piece tabs that interlock puzzle pieces together which interlocks the members. The implant can include a shuttle recess or aperture, which is shown to be oval, and preferably is scalloped and can include a shuttle member that is contained by a shelf that diverges inwardly to carry materials to the disc. The shuttle aperture can be filled with any type of material, such as porous metal or polymer, to allow for cell ingrowth or can be filled with an active agent or pharmaceutical as well as drug delivery compositions.

While not shown in FIG. 2 or FIG. 4, the clamshell can include surface grooves, such as omega recesses, that facilitate implantation and retaining the implant in a position between adjacent vertebrae.

The implant can be assembled prior to insertion, when partially inserted, or after being fully inserted. The implant can be assembled in any direction including sliding members relative to each other, such as in the direction C or D, or pressing or snap-fitting in a direction normal arrows C and D.

The elasticity of at least one portion or tab of the implant enables the tabs to enter the narrower portion of a receptacle with more velocity and low pressure, and then stress relaxes to expand within the wider portion of the receptacle expanding the tab. The tab pressure can exceed the pressure within the implant. A lower pressure at the surface of the implant can reduce frictional forces and therefore allow for easier insertion of the implant. A lower pressure at the surface of the implant can reduce frictional forces protecting the vertebral endplate from damage.

FIG. 6A shows a first portion of an implant that has some elasticity to it, so when the second portion gets inserted into it, it can actually be locked. The second portion is inserted to expand this first portion so that it will open up, and then once the tab hits the back of the receptacle, then it will close together. The second piece can be a shaft, such as for a rotatable plate, or for a cam or the like.

In one embodiment, a tapered rib portion of the implant shown in FIG. 11 arcuately expands in the direction of arrow F when compressed in the direction of arrow A. A side locking tab reversibly moves at the aperture shown allowing another portion (not shown) to move tapered rib arcuately up in the direction of arrow F. A side tab then locks in a recess on the side of the second portion to maintain the tapered rib in its expanded position. This can open the implant module for separating or decoupling from an adjacent implant module, and close the implant module for coupling with an adjacent implant module

FIG. 12B shows an implant wherein locking tab interlocks first and second implant members with a ratcheted tab. The first and second members can be disposed along a threaded wire, where the first member is compressed to the second member by turning the threaded wire. The threaded wire can then be removed from implant by further rotating the threaded wire leaving the first and second portions locked to form the implant. The threaded wire can act as for maintaining the alignment of the modules linearly, and then as the wire is pulled, the implant can advance off the wire, which can be done with a ratchet mechanism using the threads as ratchet members.

The advantage of the ratchet is that it can be in a partially locked position, and then it can be locked further in a second position or a third position, so as to be capable of providing control to the amount of angulation with respect to one portion to the second portion of the implant by incremental engagement with a corresponding ratchet receptacle. The ratcheting can be of continuous separation of ratchet members or by varying the distance between ratchet members. For example, initially the distances can be large because there will be a limited amount of force required to articulate it, and then with further insertion, the distance between the teeth can get logarithmically smaller so as to fine tune the insertion via ratcheting.

FIG. 16A illustrates a stepped cam shaft that is rotationally operable to spread two members. Steps in the cam shaft provide grooves or recesses for interlocking a shaft in a housing. The tab can be sequentially wider, so it acts as a ratchet upon insertion into a receptacle, whereas it separates, locks it, and then it separates and then locks it.

In one embodiment, the multi-membered implant can include a fixed or rotatable plate at an end, which can be configured as a bone plate for mounting to adjacent vertebrae as described in the incorporated references. The rotatable plates generally are flat or planar structures with various lengths and widths with a thickness that is usually thinner than the length or width. The thinner thickness allows the rotatable plate to fit on or in a bone with a low profile so that the body of the bone plate does not extend too far from the surface of the bone. The rotatable plate can include apertures that are configured to receive screws therethrough for affixation to a bone surface. The apertures can be of various sizes, cross-sectional profiles, and configurations (e.g., threaded or smooth) to receive various types of fasteners, such as bone screws. An embodiment of the invention provides the implant having a rotatable plate configured for attachment to a vertebra. The rotatable plate can be shaped as an oval, rectangle, or other elongated polygon. In one embodiment, the bone plate implant includes at least one aperture configured for receiving a fastener therethrough so as to fasten the implant to bone. The aperture can be located on any portion of the body, such as in a main body region or arm body region.

In one embodiment, the multi-membered implant can be configured as a bone plate and can be configured as in the incorporated references, such as in the provisional application, where the bone plate has flexible tabs and apertures associated and/or at least partially defined by the flexible tabs. The multi-membered plate can be a spinal plate embodiment, can be used for stabilizing one or more vertebrae, and can be mounted to a single vertebra or two or more adjacent vertebrae in order to stabilize and facilitate healing of factures, disc herniations, fusions, inhibition of fusions, and other common spinal plate uses. In some instances, spinal plates can be referred to as lumbar plates, anterior lumbar, or lateral plates. The rotatable plate may mount on an outside perimeter surface. However, the rotatable plate may be rotated to be embedded between the vertebrae so as to contact each vertebral surface. Also, the plates can be used to treat spinal deformities, trauma, degenerative lumbar, or the like for treatment of cervical spine applications, anterior column support, posterior applications, or any other. The plates can be used for attachment to one vertebra or to two or more adjacent vertebrae.

Any of the bodies of the implant can include a recess, hole, aperture, or other indentation that can be filled with a material, such as a porous material. This feature can be filled with any type of material, such as porous metal or polymer to allow for cell ingrowth or can be filled with an active agent or pharmaceutical as well as drug delivery compositions.

Any of the bodies of the implant, such as those with surfaces that face the adjacent vertebrae, can include surface grooves that facilitate implantation and retaining the implant in a position between adjacent vertebrae. The surface grooves can be scalloped or omega-shaped (e.g., “Ω”) recesses that provide interlocking features when receiving bone ingrowth.

The different bodies, tabs, body defining receptacles, cams, rotatable plates, or engagement members of the implant can be prepared from one or more materials that are biocompatible. The different components can be prepared from different types of materials or all from the same type of material. Examples of materials can include polymers, ceramics, composites, metals, alloys, hybrid materials, and combinations thereof, which can be biostable, biodegradable, or resorbable. The materials of the cams and main housing body can be the same or different.

In one embodiment, a kit can include the implant modules that can be assembled into a multi-modular implant. Also, the kit can include one or more fasteners, the fasteners being adapted to be received into the apertures in the housing or rotatable plate. The implant members can be prepared from the same or different biocompatible materials, such as metals, ceramics, polymers, or others.

In one embodiment, the surface of the implant can include recesses, such as a bone-contacting surface having recesses that can receive bone growth therein. The recesses can be narrower at the opening than the base, or wider at the opening than the base, or have a uniform cross-sectional profile. These bone-receiving recesses can function to lock the implant to bone when bone grows therein. Also, the recesses can be filled with polymers or other compositions that can contain bone growth factors or other active agents to promote bone growth into the bone-receiving recesses.

In one embodiment, the implant includes a rectangular plate that is rotatably attached to an end opposite of a tapered end of the implant having a housing body with a rectangular cross-sectional profile such that the plate can be in a first position with respect to the housing body such that the rectangular plate and housing body align (e.g., “_”) and rotatable to a second position where the rectangular cross-sectional profiles of the plate and housing body are at an angle, such as a 90-degree angle or to form a “+” shape or any angle between aligned and orthogonal. The plate can be configured to be a bone plate, and can have apertures for fastening to bone such as perimeter surfaces of adjacent vertebrae. Fixing the rotatable plate to a vertebra can prevent the rotatable plate or implant coupled thereto from moving. When the rotatable plate is coupled to an implant housing, once the implant device is rotated, expanded, separated, joined, articulated, pivoted, or otherwise manipulated, the rotatable plate can be attached to bone to inhibit the implant from being further rotated, expanded, separated, joined, articulated, pivoted, or otherwise manipulated.

One skilled in the art will appreciate that for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions, or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims), are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

All references, patent applications, published patent applications, and patents recited herein are incorporated herein by specific reference in their entirety: U.S. patent application Ser. No. 13/605,756, filed Sep. 6, 2012, which is a continuation-in-part of U.S. patent application Ser. No. 13/370,925 filed Feb. 10, 2012 and continuation-in-part of U.S. patent application Ser. No. 13/478,870 filed May 23, 2012, which are continuation-in-parts of U.S. patent application Ser. No. 13/199,324 filed Aug. 26, 2011 [P12], which is a continuation-in-part of U.S. patent application Ser. No. 13/065,291 filed Mar. 18, 2011 [P11], which is a continuation-in-part of U.S. patent application Ser. No. 11/804,838 filed May 21, 2007 [P8], now U.S. Pat. No. 7,909,872, which is a continuation-in-part of U.S. patent application Ser. No. 11/638,652 [P7], now U.S. Pat. No. 7,883,542 filed Dec. 12, 2006, which is a continuation-in-part of U.S. patent application Ser. No. 11/472,060 [P6], now U.S. Pat. No. 7,879,099 filed Jun. 21, 2006, which is a continuation-in-part of U.S. patent application Ser. No. 11/404,938 [P5], now U.S. Pat. No. 7,727,279 filed Apr. 14, 2006, which is a continuation-in-part of U.S. patent application Ser. No. 11/351,665 [P4] filed Feb. 10, 2006 now abandoned, which is a continuation-in-part of U.S. patent application Ser. No. 11/299,395 [P3] filed Dec. 12, 2005 now abandoned, which is a continuation-in-part of U.S. patent application Ser. No. 11/241,143 [P2] filed Sep. 30, 2005 now abandoned, which is a continuation-in-part of U.S. patent application Ser. No. 11/145,372 filed Jun. 3, 2005 now abandoned, and U.S. patent application Ser. No. 13/605,752 filed Sep. 6, 2012, which is a continuation-in-part of U.S. patent application Ser. No. 11/827,519 filed Jul. 12, 2007, which applications and patents are incorporated herein by specific reference.

1. An implant comprising:
a first implant body having one or more locking tabs; and
a second implant body having one or more locking receptacles containing the locking tabs of the first implant body so as to couple the first implant body with the second implant body.

2. The implant of claim 1, wherein the first implant body includes a plurality of locking tabs received into a plurality of locking recesses.

3. The implant of claim 1, wherein the locking tabs are interlocking tabs that have tab heads with cross-sectional profiles larger than tab stems, and the locking receptacles are interlocking receptacles with shapes that conform with the interlocking tabs.

4. The implant of claim 1, wherein the first implant body and second implant body are coupled in a sandwich format.

5. The implant of claim 1, wherein the first implant body and second implant body are coupled in a train format.

6. The implant of claim 1, wherein the first implant body and second implant body are coupled in a clamshell format.

7. The implant of claim 6, wherein the first implant body includes a top portion and bottom portion that receive the second implant body therebetween.

8. The implant of claim 1, wherein the second implant body includes one or more locking tabs received into one or more locking receptacles of the first implant body.

9. The implant of claim 1, wherein the implant includes a top surface and bottom surface both with omega-shaped recesses.

10. The implant of claim 1, comprising a third implant body coupled to one of the first or second implant body by corresponding interlocking tabs and interlocking receptacles having the interlocking tabs.

11. The implant of claim 1, comprising at least one shuttle recess extending from a top surface and/or bottom surface, the shuttle recess including a shuttle member having a porous bone or metal material.

12. The implant of claim 1, wherein the first implant body includes a material different from the second implant body.

13. The implant of claim 1, comprising an endplate having fastener apertures attached to an end of the implant.

14. The implant of claim 1, comprising a central conduit extending from a front end to a back end.

15. The implant of claim 1, wherein the second implant body has one or more interlocking tabs that are interlocked with the one or more locking tabs of the first implant body.

16. The implant of claim 1, wherein at least one of the locking tabs or locking receptacles has a chamfered end.

17. The implant of claim 1, wherein the locking tab has a slit and forms two adjacent tab portions that can be pressed together.

18. The implant of claim 1, wherein the locking tab extends from a side of a body of the first implant member and is pivotal with respect to the body.

19. The implant of claim 1, wherein the locking tab includes at least one retractable protrusion, and the locking receptacle has a receptacle for the retractable protrusion.

20. The implant of claim 1, wherein the locking tab includes at least one fixed protrusion, and the locking receptacle has a receptacle for the fixed protrusion.

21. The implant of claim 1, wherein the first implant member and second implant member are pivotally linked through a hinge.

22. The implant of claim 1, wherein the locking tab and locking receptacle include corresponding ratchet members.

23. The implant of claim 1, wherein the implant is an intervertebral implant.

24. The implant of claim 1, wherein the one or more locking receptacles is expandable in order to receive the one or more locking tabs.

25. A method of assembling the implant of claim 1, comprising:
providing the first implant member and second implant member;
introducing the locking tab into the locking receptacle; and
coupling the first implant member and second implant member by inserting a head of the locking tab into a cavity of the locking receptacle.

26. The method of claim 25, wherein the locking tab is snap-fit pressed into the locking receptacle.

27. The method of claim 25, wherein the locking tab is configured as a rail and the locking receptacle is configured as a locking slot, and the rail is slid in the slot by sliding the first implant relative to the second implant in the direction of the rail and slot.
